(19) United States
(12) Patent Application Publication
(10) Pub. No.: US 2010/0106037 A1
(43) Pub. Date: Apr. 29, 2010

(54) METHOD AND DEVICE FOR DETERMINING CARDIAC OUTPUT WITH CARBON DIOXIDE PARTIAL RE-BREATHING

(76) Inventors: Robert M. Kacmarek, Littleton, MA (US); Yandong Jiang, North Reading, MA (US); Yafen Liang, Quincy, MA (US)

Correspondence Address: QUARLES & BRADY LLP, 411 E. WISCONSIN AVENUE, SUITE 2040, MILWAUKEE, WI, 53202-4497, US

(21) Appl. No.: 12/530,397

(22) PCT Filed: Mar. 5, 2008
(86) PCT No.: PCT/US08/55866

§ 371 Date: Sep. 8, 2009

Related U.S. Application Data
(60) Provisional application No. 60/893,447, filed on Mar. 7, 2007.

(51) Int. Cl. A61B 5/029; A61M 16/06; A61M 16/20

(52) U.S. Cl. 600/526; 128/205.25; 128/205.24

(57) ABSTRACT

A method and apparatus (10) for determining cardiac output by directing airflow or gas flow (fluid) to a fluid containment structure (12) coupled to an inspiratory limb (16) of a ventilator circuit instead of the patient and enabling the patient to directly inhale fluid from the fluid containment structure and preventing the patient from inhaling through an expiratory limb (18) of the ventilator-circuit. The method and apparatus also prevents exhalation by the patient into the fluid containment structure. The method and apparatus also comprises an aid helps the patient become consistent in their breathing patterns. When the fluid containment structure fills with a volume of fluid, the aid provides an indication that the patient should inhale; when the fluid containment structure empties of fluid, the aid provides a signal that the patient should exhale. Thus, the aid provides feedback to the patient to help the patient control their ventilation pattern.

### FIELD OF THE INVENTION

This invention relates to a method and apparatus for measuring cardiac output (CO) in spontaneously breathing humans whose tracheas are not intubated.

### BACKGROUND OF THE INVENTION

Measurement of cardiac output (CO) is frequently performed to guide hemodynamic management of critically ill patients. Since the introduction of the pulmonary artery catheter (PAC) by Swan and Ganz in 1970, the thermodilution technique using PAC has gained widespread acceptance, and is considered the clinical gold standard for the measurement of CO. However, PACs are invasive and have been associated with serious errors and complications. The ideal method for measurement of CO should be noninvasive, accurate, reliable, and continuous.

The noninvasive cardiac output monitor (NICO) manufactured by Respironics, Inc., Wallingford, Conn. uses the differential carbon dioxide (CO₂) Fick partial rebreathing technique to determine CO non-invasively. This technique compares measurements of exhaled CO₂ obtained during a non-rebreathing period with those obtained during a subsequent re-breathing period. The ratio of the change in end-tidal carbon dioxide partial pressure (PETCO₂) and carbon dioxide elimination (VCO₂) after a brief period of partial rebreathing provides the noninvasive estimate of CO.

One problem with this technique, however, is that using NICO to determine CO requires tracheal intubation and mechanical ventilation in order to insure a constant minute ventilation. If minute ventilation varies, the CO measurement with the NICO monitor is inaccurate, due to inconsistent CO₂ removal. As a result, many patients whose tracheas are not intubated are excluded from using the current NICO technique to obtain CO.

It would therefore, be desirable, to provide a method and device to use a NICO monitor to measure CO in humans whose tracheas are not intubated.

The foregoing features of this invention, as well as the invention itself, may be more fully understood from the following description of the drawings in which:

### SUMMARY OF THE INVENTION

A method for determining cardiac output of a patient comprising directing airflow or gas flow to a fluid containment structure coupled to a ventilator circuit instead of to the patient by a one-way valve and enabling the patient to directly inhale air or gas from the fluid containment structure by a operating a second one-way valve in concert with the first one-way valve. The method further includes preventing inhalation by the patient from an expiratory limb of the ventilator circuit by a third one-way valve and preventing exhalation into the fluid containment structure by a one-way valve.

A method for providing visual feedback to a patient to help the patient control their ventilation pattern includes providing a fluid containment structure which the patient looks at and when the patient sees the fluid containment structure filled with a volume of fluid, the patient inhales. When the patient sees the fluid containment structure empty of fluid, the patient exhales. The fluid containment structure thus provides visual feedback to a patient to help the patient control their ventilation pattern The fluid containment structure (which may be provided as a balloon, bellows or syringe, for example) acts as a metronome which aids patients in becoming consistent in their breathing patterns. In one exemplary embodiment, the fluid containment structure is provided as a balloon and the patient looks at the balloon; when the patient sees the balloon filled with a volume of fluid (e.g. by observing the size and shape of the balloon), the patient inhales. When the patient sees the balloon empty of fluid (e.g. by observing the size and shape of the balloon), the patient exhales. Thus, the balloon provides visual feedback to a patient to help the patient control their ventilation pattern. This affects both the rate and volume which the patient breathes). This technique provides a visual aid which the patient can utilize to see how fast they are breathing. In other embodiments, audio and/or mechanical aids can be used either in conjunction with or in place of a visual aid. For example, an audio aid can be provided such that a sound is made when the fluid containment structure is either filled or empty. Or an mechanical aid can be provided such that a vibration occurs when the fluid containment structure is either filled or empty. Other visual, audio or mechanical aids (or combinations of such aids) not specifically described or mentioned herein will be readily apparent to those of ordinary skill in the art. An airflow direction system may be provided by three one-way valves operated in concert to appropriately direct airflow to/from the patient.

A device includes an oral-nasal face mask, a ventilator having a first port, a ventilator circuit in fluid communication with said oral-nasal face mask, said ventilator circuit having an inspiratory limb having a port coupled to said ventilator and an expiratory limb having a port, a fluid containment structure having a first port in fluid communication with the inspiratory limb of said ventilator circuit and having a second port, an airflow direction system in fluid communication with the inspiratory and expiratory limbs of said ventilator circuit, said airflow direction system coupled to allow gas to only fill the fluid containment structure at a first point in time and to allow a patient to breathe gas only from the a fluid containment structure and a non-invasive cardiac output (NICO) monitor coupled to the ventilator circuit. With this arrangement, a device for determining cardiac output of a patient is provided. The device is non-invasive. In one embodiment, the ventilator circuit comprises a y-piece having a first path corresponding to the inspiratory limb of the ventilator circuit, a second path corresponding to the expiratory limb of the ventilator circuit and a third path adapted to couple to a rebreathing valve. The airflow direction system can be provided from three valves. The first valve disposed in the inspiratory limb of the ventilator circuit and having a first port in fluid communication with the ventilator and a second port in fluid communication with a first port of the fluid containment structure. The second valve disposed in the inspiratory limb of the ventilator circuit and having a first port in fluid communication with a second port of the fluid containment structure and a second port in fluid communication with the third path. The third valve disposed in the expiratory limb of the ventilator circuit. The first and second valves open in alternated order, but not at the same time. The first valve is operable to allow a patient to inhale gas only from the fluid containment structure, the second valve is operable to deliver a targeted volume of gas to the fluid containment structure only, but not to the patient. The third valve is operable to prevent the patient from inhaling gas from the expiratory limb of the ventilator circuit.

A method includes applying a ventilator circuit to a patient's natural airway using an oral-nasal face mask, delivering mechanical ventilation with a specific predetermined tidal volume and ventilatory rate and measuring cardiac output (CO) with a non-invasive cardiac output (NICO) monitor using a NICO technique. In one embodiment, delivering mechanical ventilation with a specific predetermined tidal volume and ventilatory rate comprises operating a first valve in fluid communication with and positioned between the ventilator and the fluid containment structure to fill the fluid containment structure with gas and to allow a patient to inhale gas only from the fluid containment structure, operating a second valve in fluid communication with and positioned in the inspiratory limb of the ventilator circuit between the fluid containment structure and the Y-piece to deliver a targeted volume of gas to the fluid containment structure only, but not to the patient wherein the first and second valves open in alternating order, but not at the same time and operating a third valve in fluid communication with the expiratory limb to prevent the patient from inhaling gas from the expiratory limb of the ventilator circuit. In one embodiment, the patient breathes normally through the oral-nasal face mask and the non-invasive cardiac output (NICO) monitor without attachment to a ventilator until their ventilatory pattern is stable. The patient's tidal volume, respiratory rate and minute ventilation are then measured and the average tidal volume and respiratory rate are computed. The method further includes attaching the ventilator to the NICO monitor and operating the ventilator to provide volume targeted mechanical ventilation to a fluid containment structure and holding the patient's minute volume and carbon dioxide (CO₂) elimination constant. In one embodiment, the ventilator is operated to provide volume targeted mechanical ventilation to a fluid containment structure with the ventilator set at or slightly above the patient's average tidal volume and respiratory rate. In one embodiment, a plurality of valves are operated in concert to fill the fluid containment structure with gas and to allow a patient to inhale gas only from the fluid containment structure and to prevent the patient from inhaling gas from any other portion of the ventilator circuit.

A method comprises ventilating a fluid containment structure in fluid communication with an inspiratory limb of a ventilator circuit and having a patient passively inspire a fixed volume of gas only from the fluid containment structure wherein the fluid containment structure prevents ventilator pressure from being applied directly to the patient's airway such that a ventilatory pattern is maintained consistent and a cardiac output of the patient is prevented from being altered by the application of positive pressure.

A method for determining cardiac output of a patient includes operating an airflow direction system to allow a fluid containment structure to be filled with a substantially predetermined volume of gas, providing an indication that the fluid containment structure is filled with the substantially predetermined volume of gas and in response to the indication that the fluid containment structure is filled with the substantially predetermined volume of gas, allowing the patient to inhale the volume of gas from the fluid containment structure. The method further includes, in response to an indication that the substantially predetermined volume of gas has been emptied from the fluid containment structure, allowing the patient to exhale. The fluid containment structure provides an indicator of when it is filled with and empty of gas and also provides the patient with feedback such that patients can easily keep up with the indicator of when to inhale and exhale and thus maintain a substantially constant tidal volume constant. The indicator may be provided as one or all of a visual, audio and/or mechanical aid. The indicator(s) can be used either in conjunction with each other or individually. For example, an audio aid can be provided such that a sound is made when the fluid containment structure is either filled or empty. Or an mechanical aid can be provided such that a vibration occurs when the fluid containment structure is either filled or empty. Other visual, audio or mechanical aids (or combinations of such aids) not specifically described or mentioned herein will be readily apparent to those of ordinary skill in the art.

### BRIEF DESCRIPTION OF THE DRAWINGS

FIG. 1 is a schematic diagram of a device used to measure cardiac output (CO) with a noninvasive cardiac output (NICO) monitor and which includes three one way valves and a fluid containment structure; and

FIG. 2 is an exemplary embodiment of a NICO system having a Y-piece from a ventilator circuit and facemask coupled thereto.

### DETAILED DESCRIPTION OF THE PREFERRED EMBODIMENTS

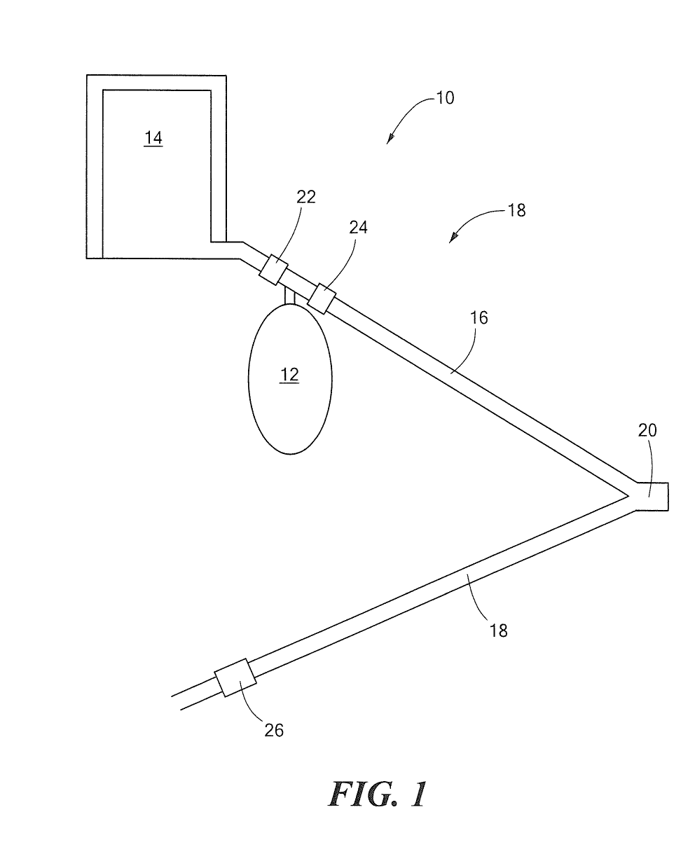

Referring now to FIG. 1, a device 10 to measure cardiac output (CO) in humans whose tracheas are not intubated includes a fluid containment structure or reservoir 12. It should be appreciated that reservoir 12 may be provided as any container with high compliance and low resistance. In one exemplary embodiment, a balloon having a capacity of 1 to 3 liters and which could be extended to 10 liters with pressure no higher that 40 cm of water was used. The fluid containment structure 12 may, for example, be provided as a balloon, a bellows, or a syringe. In one embodiment, a balloon comprised of a compliant material may be used. Such balloons are commercially available. In another exemplary embodiment, a balloon capable of holding three litters of gas at ambient pressure and which can be extended up to ten liters with maximal pressure less than forty centimeters of water (cm H₂O) may be used. In another embodiment, a highly compliant balloon may be used. As used herein, the term “highly compliant balloon” refers to a balloon having compliance of greater than or equal to about 300 ml/cm H₂O. Such balloons are commercially available.

In alternate embodiments, a syringe can be used instead of a balloon. It should be appreciated, of course, that the fluid containment structure may be provided as any type of fluid reservoir including but not limited to the above-mentioned high compliance balloon or the syringe.

The fluid containment structure 12 is in fluid communication with a ventilator 14 through an inspiratory limb 16 of a ventilator circuit. The ventilator circuit can be provided as a double-limb ventilator circuit or as a single limb ventilator circuit with an exhalation valve. The ventilator circuit (16, 18, 20) includes an inspiratory limb 16, an expiratory limb 18 and also a Y piece 20 which is coupled to a conventional NICO sensor (not shown in FIG. 1). The NICO sensor is then applied to a patient's natural airway (not sown in FIG. 1) using an oral or nasal or oral-nasal face mask (not shown in FIG. 1). In some alternate embodiments, a mouth piece and nose clip (not shown in FIG. 1) may be used in place of the oral-nasal face mask. Such oral-nasal facemasks (or mouth piece and nose clip) are well known to those of ordinary skill in the art and are commercially available.

Mechanical ventilation is delivered with a specific predetermined tidal volume and ventilatory rate. A NICO monitor is attached to the ventilator circuit in its usual manner and CO is measured by the well-known NICO technique.

It should be appreciated that the ventilation structure and technique described in conjunction with FIG. 1 allows one to obtain constant minute ventilation without tracheal intubation.

In operation, the patient first breathes normally through the mask and NICO monitor without attachment to the ventilator (i.e. the ventilator and the ventilator circuit are not initially attached to the NICO monitor). This mode of operation continues until the patient's ventilatory pattern is stable (less than about a 10% variation in tidal volume and minute volume). When the patient breathes comfortably and the minute ventilation is fairly constant (less than about a 10% variation), their tidal volume, respiratory rate and minute ventilation are measured. The average tidal volume is then calculated using the minute ventilation volume divided by the measured respiratory rate (breaths/minute). The average respiratory rate is calculated by averaging the patient's ventilation rate during the initial breathing period (breaths/minute).

The ventilator and the ventilator circuit are then attached to the NICO monitor and assisted volume targeted mechanical ventilation is provided. It should be emphasized that the ventilator is set at or slightly above the patient's average tidal volume and respiratory rate during this process (less than or equal to about 15% greater) to insure the patient's end-tidal CO₂ is decreased in the range of about 2% to about 12% with a decrease in the range of about 5% to about 10% being preferred. By appropriately setting the ventilator, the patient's minute volume is held substantially constant and their carbon dioxide (CO₂) elimination is constant. The constant CO₂ elimination is accomplished by constant minute ventilation, which is accomplished by appropriate ventilator settings and coaching by a health care provider. The fluid containment structure (e.g. a balloon of highly compliant material) coupled to the inspiratory limb of the ventilator circuit prevents ventilator pressure from being applied directly to the patient's airway and thus, the patient airway is not exposed to the positive pressure generated by the ventilator. That is, the ventilator ventilates the fluid containment structure (e.g. the balloon, bellows, or syringe) and the patient passively inspires the fixed volume from the fluid containment structure. Accordingly, any structure which provides this function (i.e. allows a patient to passively inspire a fixed volume) can serve as the fluid containment structure.

There is a one-way valve 22 (first valve) in fluid communication with and positioned between the ventilator 14 and the fluid containment structure 12 (e.g. up-stream from the fluid containment structure and between the fluid containment structure and the ventilator), which allows the patient to inhale the gas from the fluid containment structure only (i.e. not from the ventilator).

A second one-way valve 24 in fluid communication with and positioned in the inspiratory limb between the fluid containment structure and the Y-piece 20 (e.g. down stream from the fluid containment structure and between the fluid containment structure and the Y-piece) allows the ventilator 14 to deliver a targeted volume of gas to the fluid containment structure only, but not to the patient. These two valves (i.e. valves 22, 24) open in alternated order, but not at the same time. This prevents the patients' cardiac output from being altered by the application of positive pressure. Therefore, the tidal volume per breath, the respiratory rate (frequency of breathing) and the minute CO₂ removal are kept constant just as if the person's trachea was intubated and their lungs mechanically ventilated. CO measurement with the NICO is also conducted just as in the patient who is mechanically ventilated.

As stated above, valve 22 is placed between the ventilator and the fluid containment structure. This valve is a passive valve, with opening pressure in the range of about 15 to about 30 cm H₂O. The purpose of this valve is to prevent the patient from inhaling additional gas volume directly from the ventilator. It works as follows: when the ventilator delivers gas, the airway pressure increases; when pressure generated by the ventilator reaches the opening pressure in the range of greater than about 15 to about 30 cm H₂O, the valve opens, and gas will go to the fluid containment structure but not to the patient due to the second one way valve 24. The fluid containment structure has less resistance to air flow than the patient's lungs which is insured by the one way valve 24. Thus, air will be delivered only to the fluid containment structure but not to the patient.

When the ventilator stops delivering gas, the ventilator pressure drops below the opening pressure of the one-way valve 22, then valve 22 will be closed. Then the patient is instructed to inspire gas passively from the fluid containment structure (e.g. the balloon). Since the opening pressure of the valve is much higher then the inspiratory pressures generated by quiet breathing, the valve prevents the patient from inhaling any additional gas except that in the fluid containment structure. This prevents the patients' cardiac output from being altered by the application of positive pressure. Therefore, the CO measurement with the NICO is conducted just as if the person's trachea was intubated and their lungs mechanically ventilated.

The second valve 24 is placed between the balloon and the Y piece, it is a passive valve with opening pressure of about 0.5 to about 2.0 cm H₂O with about 1.0 cm H₂O being preferred. When the patient inhales, the negative pressure generated in the airway will open the second valve 24. This valve prevents backflow of gas into the inspiratory limb (e.g. into the limb having the balloon coupled thereto) of the circuit when the patient exhales. The second valve 24 also prevents the gas delivered by the ventilator from going to the patient directly, since the pressure required to open the valve is higher than the pressure in the fluid containment structure.

A third valve 26 is placed in fluid communication with the expiratory limb 18. This valve prevents the patient from inhaling gas from the expiratory limb of the ventilator circuit. This valve should have an opening pressure in the range of about 0.5 to about 2.0 cm H₂O with about 1.0 cm H₂O being preferred.

This technique has been tested on 20 healthy volunteers. The preliminary study has demonstrated the feasibility of using this technique and the ability to maintain a constant respiratory rate and inspired gas volume. The cardiac output determined by this technique in these volunteers was highly reproducible. It has also been found that this technique is very comfortable for the volunteers and easy to coach.

In particular, the balloon (or some other fluid containment structure) acts as a metronome which aids patients in becoming consistent in their breathing patterns. This is accomplished as follows: the patient looks at the balloon (or other fluid containment structure) and when the patient sees the fluid containment structure filled with a volume of fluid, the patient inhales. When the patient sees the fluid containment structure empty of fluid, the patient exhales. Thus, the balloon (or other fluid containment structure) provides visual feedback to the patient to help the patient control their ventilation pattern. This affects both the breathing rate and breathing volume of the patient). The system and techniques described herein thus provide a visual aid which a patient can utilize to see how fast they are breathing.

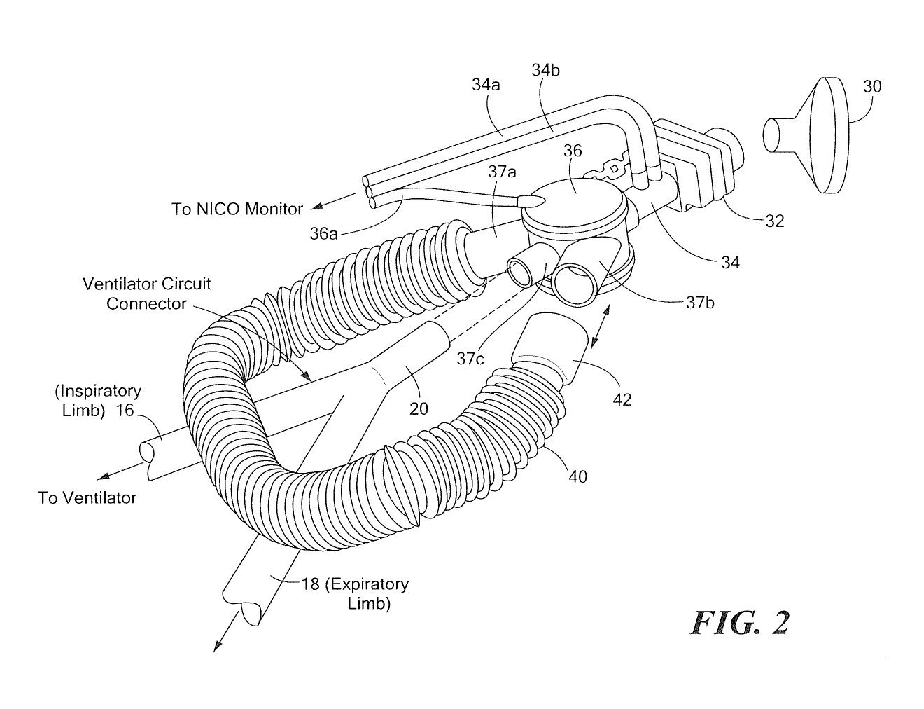

Referring now to FIG. 2, the portion of the system for measuring cardiac output (CO) in humans whose tracheas are not intubated includes a facemask 30, or an oral piece or a nasal mask coupled to a CO₂ sensor 32 which in turn is coupled to a flow sensor 34. The flow sensor 34 is coupled to a disposable automatic rebreathing valve 36. The two tubes 34a, 34b emanating from the flow sensor 34 and the tube 36a emanating from the rebreathing valve are coupled to the NICO monitor (not shown in FIG. 2). In one embodiment, the CO₂ sensor maybe provided as the type manufactured by Respironics and marketed under the tradename CAPNOSTAT®.

A NICO loop 40 has a first end coupled to a first port 37a of the disposable automatic rebreathing valve 36. A second end of the NICO loop is adapted to be coupled to a second port 37b of the disposable automatic rebreathing valve 36 through a loop drainage coupler 42. A Y-piece 20 has a first port adapted to be coupled to a third port 37c of the disposable automatic rebreathing valve 36, a second port (i.e. a first one of the limbs of the Y) adapted to be coupled to the inspiratory limb 16 of a ventilator circuit and a third port (i.e. a second one of the limbs of the Y) adapted to be coupled to the expiratory limb 18 of a ventilator circuit. The CO₂ sensor, flow sensor, disposable automatic rebreathing valve and the NICO loop form a portion of a NICO monitor. Thus, the NICO circuit is coupled to the ventilator circuit through the Y piece, which is also a part of the ventilator circuit while the flow sensor is a part of the NICO rebreathing circuit.

The purpose of the flow sensor 34 is to monitor respiratory parameters such as tidal volume and respiratory rate. This information will allow an operator of the system (e.g. a health care practitioner) to set up the same or slightly higher tidal volume and respiratory rate for the ventilator to deliver to the fluid containment structure patient. This part of the NICO system also measures pressure and exhaled CO₂.

Having described preferred embodiments of the invention it will now become apparent to those of ordinary skill in the art that other embodiments incorporating these concepts may be used. Accordingly, it is submitted that the inventions described herein should not be limited to the described embodiments but rather should be limited only by the spirit and scope of the appended claims.

1. A device comprising:
an oral-nasal face mask;
a ventilator having a first port;
a ventilator circuit in fluid communication with said oral-nasal face mask, said ventilator circuit having an inspiratory limb having a port coupled to said ventilator and an expiratory limb having a port;
a fluid containment structure having a first port in fluid communication with the inspiratory limb of said ventilator circuit and having a second port;
an airflow direction system in fluid communication with the inspiratory and expiratory limbs of said ventilator circuit, said airflow direction system coupled to allow gas to only fill the fluid containment structure at a first point in time and to allow a patient to breathe gas only from the a fluid containment structure; and
a non-invasive cardiac output (NICO) monitor coupled to the ventilator circuit;

2. The device of claim 1 wherein said ventilator circuit comprises a y-piece having a first path corresponding to the inspiratory limb of the ventilator circuit, a second path corresponding to the expiratory limb of the ventilator circuit and a third path adapted to couple to a rebreathing valve.

3. The device of claim 1 wherein said airflow direction system comprises:
a first valve disposed in the inspiratory limb of the ventilator circuit, said first valve having a first port in fluid communication with the ventilator and a second port in fluid communication with a first port of the fluid containment structure;
a second valve disposed in the inspiratory limb of the ventilator circuit, said second valve having a first port in fluid communication with a second port of the fluid containment structure and a second port in fluid communication with the third path; and
a third valve disposed in the expiratory limb of the ventilator circuit.

4. The device of claim 1 wherein
said first valve is in fluid communication with and positioned between the ventilator and the fluid containment structure and operable to allow a patient to inhale gas only from the fluid containment structure;
said second valve is in fluid communication with and positioned in the inspiratory limb of said ventilator circuit between the fluid containment structure and the Y-piece and is operable to deliver a targeted volume of gas to the fluid containment structure only, but not to the patient wherein the first and second valves open in alternated order, but not at the same time; and
said third valve is in fluid communication with the expiratory limb 18 and operable to prevent the patient from inhaling gas from the expiratory limb of the ventilator circuit.

5. A method comprising:
applying a ventilator circuit to a patient's natural airway using an oral-nasal face mask.
delivering mechanical ventilation with a specific predetermined tidal volume and ventilatory rate;
measuring cardiac output (CO) with a non-invasive cardiac output (NICO) monitor using a NICO technique.

6. The method of claim 5 wherein delivering mechanical ventilation with a specific predetermined tidal volume and ventilatory rate comprises;
operating a first valve in fluid communication with and positioned between the ventilator and the fluid containment structure to fill the fluid containment structure with gas and to allow a patient to inhale gas only from the fluid containment structure;
operating a second valve in fluid communication with and positioned in the inspiratory limb of the ventilator circuit between the fluid containment structure and the Y-piece to deliver a targeted volume of gas to the fluid containment structure only, but not to the patient wherein the first and second valves open in alternating order, but not at the same time; and
operating a third valve in fluid communication with the expiratory limb to prevent the patient from inhaling gas from the expiratory limb of the ventilator circuit.

7. The method of claim 6 further comprising monitoring respiratory parameters of a patient.

8. A method comprising:
having a patient breath normally through an oral-nasal face mask and a non-invasive cardiac output (NICO) monitor without attachment to a ventilator until their ventilatory pattern is stable;
measuring the patient's tidal volume, respiratory rate and minute ventilation;
computing the average tidal volume and respiratory rate;
attaching the ventilator to the NICO monitor and operating the ventilator to provide volume targeted mechanical ventilation to a fluid containment structure; and
holding the patient's minute volume and carbon dioxide (CO₂) elimination constant.

9. The method of claim 8 further wherein operating the ventilator to provide volume targeted mechanical ventilation to a fluid containment structure comprises operating the ventilator to provide volume targeted mechanical ventilation to a fluid containment structure with the ventilator set at or slightly above the patient's average tidal volume and respiratory rate.

10. The method of claim 8 further wherein operating the ventilator to provide volume targeted mechanical ventilation to a fluid containment structure comprises operating a plurality of valves to fill the fluid containment structure with gas and to allow a patient to inhale gas only from the fluid containment structure and to prevent the patient from inhaling gas from any other portion of the ventilator circuit.

11. The method of claim 8 further comprising monitoring respiratory parameters of the patient.

12. A method comprising:
ventilating a fluid containment structure in fluid communication with an inspiratory limb of a ventilator circuit; and
having a patient passively inspire a fixed volume of gas only from the fluid containment structure wherein the fluid containment structure prevents ventilator pressure from being applied directly to the patient's airway such that a ventilatory pattern is maintained consistent and a cardiac output of the patient is prevented from being altered by the application of positive pressure.

13. The method of claim 12 wherein ventilating a fluid containment structure comprises ventilating one of: a balloon, a syringe and a bellows.

14. A method for determining cardiac output of a patient, the method comprising:
operating an airflow direction system to allow a fluid containment structure to be filled with a substantially predetermined volume of gas;
providing an indication that the fluid containment structure is filled with the substantially predetermined volume of gas; and
in response to the indication that the fluid containment structure is filled with the substantially predetermined volume of gas, allowing the patient to inhale the volume of gas from the fluid containment structure.

15. The method of claim 14 further comprising:
providing an indication that the substantially predetermined volume of gas has been emptied from the fluid containment structure; and
in response to the indication that the substantially predetermined volume of gas has been emptied from the fluid containment structure, allowing the patient to exhale.

16. The method of claim 14 wherein providing an indication that the fluid containment structure is filled with the substantially predetermined volume of gas comprises providing a visual indication that the fluid containment structure is filled with the substantially predetermined volume of gas.

17. The method of claim 16 wherein the fluid containment structure corresponds to a balloon and the visual indication of the fluid containment structure being filled with the substantially predetermined volume of gas is provided by the shape and size of the balloon.

18. The method of claim 17 wherein the visual indication of the fluid containment structure not being filled with gas is provided by the shape and size of the balloon.

19. The method if claim 14 wherein the fluid containment structure provides visual feedback of when it is filled with and empty of gas and also provides the patient with a visual target such that patients can easily keep up with the visual target and maintain a substantially constant tidal volume constant.
